(19) United States
(12) Patent Application Publication
(10) Pub. No.: US 2006/0182818 A1
(43) Pub. Date: Aug. 17, 2006

(54) Transdermal patch and treatment for pain and discomfort

(76) Inventor: Yon Doo Ough, Beloit, WI (US)

Correspondence Address: Raymond W. Campbell, 2350 Lathers Rd., Beloit, WI, 53511, US

(21) Appl. No.: 11/059,857

(22) Filed: Feb. 17, 2005

(51) Int. Cl. A61K 36/18; A61K 31/20

(52) U.S. Cl. 424/725; 514/558

(57) ABSTRACT

A patch and a treatment method for the treatment of human discomforts includes transdermal administration of components from soap in the area of discomfort. The patch includes a permeable layer placed against the person, and an impermeable outer cover, and may include tails by which the patch is attached to the person. Fragments of soap or a scent extract from soap are held in close proximity to the area being treated.

### FIELD OF THE INVENTION

The present invention generally relates to the treatment of physical discomforts, and, more specifically, to devices applied and methods to treat the sources of physical discomforts, such as pain, muscle cramps and spasms, and other ailments.

### BACKGROUND OF THE INVENTION

Effective treatment of pain and discomfort of various types can improve quality of life conditions significantly for individuals who experience the pain or discomfort. Some individuals experience event-specific pain from injury or surgery. Others experience recurring pain, which may be somewhat continuous, or may involve repetitious intervals of pain following periods that are more or less pain-free. Chronic conditions, such as irritable bowel syndrome, can be intermittent, but significant when the symptoms are manifested. Surgeries and injuries can result in long periods of pain for some individuals. Menstrual cramps can cause days of significant discomfort on a monthly basis for many years. Various back conditions can cause almost constant pain and discomfort.

Intense pain or discomfort can be debilitating. Even less intense pain or discomfort, if frequent, can alter an individual's life-style and activities significantly. Medical professionals and practitioners from early times have concerned themselves with finding effective treatments for pain, discomfort and other conditions that are debilitating to greater and lesser degrees.

Treatment protocols have changed and evolved. Cultural differences in the treatment of pain and discomfort have begun to breakdown as medical professionals look for effective treatments for various patients experiencing various different types of conditions. Drug therapies are used widely and are effective for many conditions. However, some patients and some conditions do not respond well to drug treatments. Holistic medicine treatments, acupuncture, various folk medicine remedies, mind-control and other treatments all have been used effectively by some individuals. Often, the effectiveness can not be explained medically or scientifically, even though the effectiveness of the treatment is clearly evident and documented by the response of the patient to the treatment. One such treatment has suggested that a bar of soap be placed near the legs while sleeping to prevent the onset of leg cramps while sleeping.

One of the difficulties in treating pain and discomfort is that the source varies, and in some situations may not be well understood. Athletes often experience recurring pain and discomfort from intense use or over-use of muscles. These and other similar conditions are often referred to as “muscle pain”. For example, distance runners of all skill levels often experience chronic calf, thigh and/or hamstring muscle pain and discomfort. In addition to pain, these conditions can be manifested by muscle cramps.

Many females regularly experience various degrees of pain, cramping and other discomfort for several days in their menstrual cycles. Conditions such as irritable bowel syndrome can be manifested by prolonged diarrhea, constipation or excessive flatulence, with associated pain, discomfort and cramping. The causes are not well understood and effective treatments are not readily available for all who suffer the symptoms.

Analgesic treatments can be used to mask pain and discomfort, but are effective for only a limited duration of time and therefore must be repeated to provide continuous relief. Muscle relaxants provide some relief for muscle spasms, but also must be repeated. Continuous, prolonged use of drugs can result in undesirable side effects, including a build up of tolerance to the drug, requiring increased dosages for long term treatment. Addiction can occur rapidly, and even predictably with some drugs, requiring very careful management of the use of the drug, and even withdrawal treatments when the drug is no longer needed. Other side effects can include possible organ functional changes and/or damage, mental and personality changes, changes to sensory perceptions and the like.

What is needed is a safe and effective treatment for a variety of pains, discomforts, cramps and other maladies that is easy to use and is suitable to be self-administered.

### SUMMARY OF THE INVENTION

The present invention provides a treatment for human discomfort, including pain cramps and other conditions that includes transdermal transmission of scent in the afflicted area. Common soap suitable for personal hygiene and made from sodium hydroxide is one effective treatment.

In one aspect thereof, the present invention provides a method for treating human physical discomfort with steps of locating the area of discomfort, obtaining a substance that emits the scent of the product from the saponification of fat and alkali, placing the substance over the area of discomfort, and maintaining the substance on the area of discomfort.

In another aspect thereof, the present invention provides a patch for the treatment of human physical discomfort with an inner layer adapted for placement in close proximity to human skin and a substance in the inner layer emitting the scent of soap made from fat and sodium hydroxide. An outer layer of material is provided on the inner layer for protecting the inner layer and the substance.

In a further aspect thereof, the present invention provides a method for treating human physical discomfort. The method steps include identifying on a person the area in which discomfort is experienced, administering the scent of common soap transdermally to the area of discomfort, and continuing the transdermal administration of the scent of soap for regular prolonged intervals until the discomfort subsides.

In a still further aspect thereof, the present invention provides a method for treating muscle cramps and muscle spasms with steps of identifying on a person the area in which muscle cramps or spasms are experienced, administering the scent of common soap transdermally to the area of muscle cramps or spasms; and continuing the transdermal administration of the scent of soap for regular prolonged intervals until the cramps subside.

In yet a further aspect thereof, the present invention provides a method for treating pain with steps of identifying on a person the area in which pain is experienced; administering the scent of common soap transdermally to the area of pain; and continuing the transdermal administration of the scent of soap for regular prolonged intervals.

In yet another aspect thereof, the present invention provides a method for treating menstrual cramps with steps of administering the scent of common soap transdermally to the lower abdomen; and continuing the transdermal administration of the scent of soap for regular prolonged intervals.

In still another aspect thereof, the present invention provides a method for relieving the symptoms of irritable bowel syndrome, with steps of administering the scent of common soap transdermally to the lower abdomen; and continuing the transdermal administration of the scent of soap for regular prolonged intervals.

In still another aspect thereof, the present invention provides a method for treating myofascial pain (“trigger point” pain) with steps of identifying on a person the area in which the pain is experienced; administering the scent of common soap transdermally to the area of pain; and continuing the transdermal administration of the scent of soap for regular prolonged intervals.

In a yet still further aspect thereof, the present invention provides a method for treating pain from a pinched nerve, with steps of identifying on a person the area in which the pinched nerve has occurred; administering the scent of common soap transdermally to the area identified; and continuing the transdermal administration of the scent of soap for regular prolonged intervals.

In other aspects thereof, the present invention provides methods for treating pain, including myofascial pain (“trigger point” pain), pain from spinal stenosis, bulging discs and scar tissue. The method steps include identifying the area of pain in the scar or along the spine; obtaining a substance including at least one of soap made from fat and sodium hydroxide, a scent oil from soap made from fat and sodium hydroxide and products occurring from the saponification of fat and alkali; placing the substance in close proximity to the area of painful; and maintaining the substance in close proximity to the area for at least about six hours in a twenty-four hour period.

In a still further aspect thereof, the present invention provides a method for treating discomfort in a person, including the treatment of pain, muscle cramps and muscle spasms. The method has steps of identifying the area of discomfort; and obtaining at least one of soap made from fat and sodium hydroxide and a scent oil from soap made from fat and sodium hydroxide; placing it in close proximity to the area of discomfort; and maintaining it in close proximity to the area of discomfort for at least about six hours in a twenty-four hour period.

In a still further aspect thereof, the present invention provides a method for treating diarrhea and intestinal gas, including those caused by irritable bowel syndrome. Steps of the method include obtaining a substance including at least one of soap made from fat and sodium hydroxide, a scent oil from soap made from fat and sodium hydroxide and products occurring from the saponification of fat and alkali; placing the substance in close proximity to the lower abdomen; and maintaining the substance in close proximity to the lower abdomen for at least about six hours in a twenty-four hour period.

In a still further aspect thereof, the present invention provides a method for treating muscle stiffness, including stiffness of neck muscles. The method includes steps of identifying the area of muscle stiffness; obtaining a substance including at least one of soap made from fat and sodium hydroxide, a scent oil from soap made from fat and sodium hydroxide and products occurring from the saponification of fat and alkali; placing the substance in close proximity to the area of muscle stiffness; and maintaining the substance in close proximity to the area of muscle stiffness for at least about six hours in a twenty-four hour period.

Other features and advantages of the invention will become apparent to those skilled in the art upon review of the following detailed description, claims and drawings in which like numerals are used to designate like features.

### BRIEF DESCRIPTION OF THE DRAWINGS

FIG. 1 is a plan view of a patch suitable for treatments in accordance with the present invention;

FIG. 2 is a cross-sectional view of a patch similar to that shown in FIG. 1, the cross-section having been taken along line 2-2 as shown on the patch of FIG. 1; and

FIG. 3 is a cross-sectional view similar to FIG. 2, but illustrating a further embodiment of the present invention.

Before the embodiments of the invention are explained in detail, it is to be understood that the invention is not limited in its application to the details of construction and the arrangements of the components set forth in the following description or illustrated in the drawings. The invention is capable of other embodiments and of being practiced or being carried out in various ways. Also, it is understood that the phraseology and terminology used herein are for the purpose of description and should not be regarded as limiting. The use herein of “including”, “comprising” and variations thereof is meant to encompass the items listed thereafter and equivalents thereof, as well as additional items and equivalents thereof.

### DETAILED DESCRIPTION OF THE PREFERRED EMBODIMENT

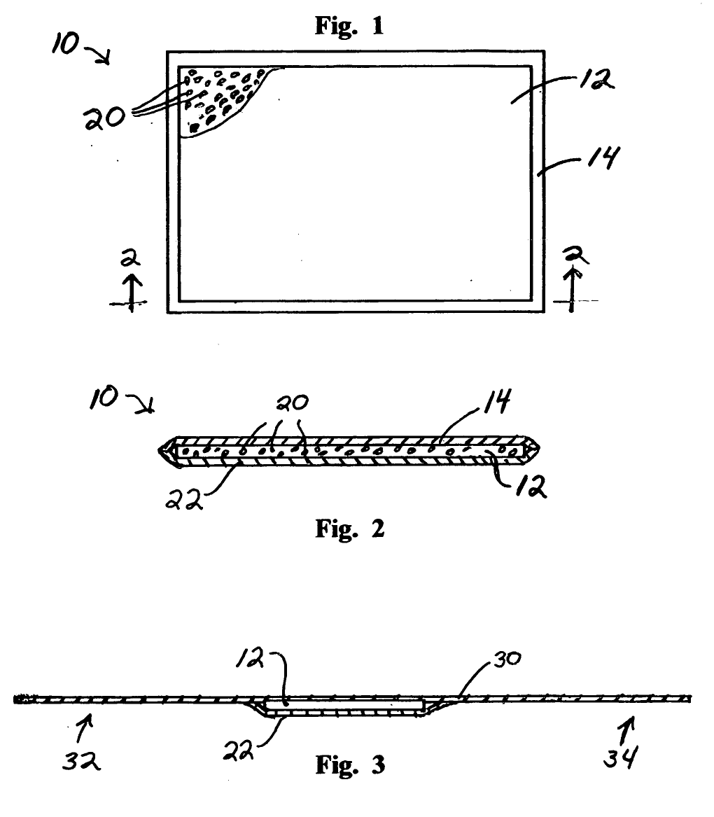

Referring now more specifically to the drawings and to FIG. 1 in particular, numeral 10 designates a transdermal patch in accordance with the present invention. Transdermal patch 10 includes a treatment layer 12 and a cover 14. Patch 10, including layer 12 and cover 14, thereof can be provided in a variety of shapes and sizes for application and use on different parts of the body.

Treatment layer 12 is a pad of cotton gauze or other material suitable for application on human skin. Gauze layer 12 is provided with a substance 20 therein having the scent of common soap, which may be a scent oil extract of common soap, or fragments, chips, shavings or other small pieces of common soap dispersed through, along and against gauze layer 12. For simplicity and clarity in the drawings, substance 20 is illustrated only in a corner region of gauze layer 12; however, it should be understood that substance 20 is relatively evenly distributed throughout gauze layer 12, in the preferred arrangement. Further, for clarity, only some, and not all of the illustrated substance 20 elements are designated with a reference numeral in FIG. 1

As used herein, including in the claims, the term “common soap” is meant to include simple or “pure” soaps made from fat and an alkali in a standard saponification or hydrolysis reaction. Soaps made from sodium hydroxide and fat are preferred. While soaps including other additives can be used, a simple or substantially “pure” soap consisting primarily of the product resulting from the saponification of a fat and alkali is preferred, without excessive amounts of perfumes, colorants, germicides, lotions or other conditioners having been added. Hand soap of a bar type manufactured by Procter & Gamble marketed under the name “Ivory” (Original Blend) has been used effectively in the present invention. Soaps made from fat and other alkalis, such as potassium hydroxide may also be suitable; however, the aforementioned “Ivory” soap has been found particularly effective.

Soaps of the type used for hand and human body washing are preferred. Such soaps have been found to work effectively in relieving pain, cramps, spasms and a variety of other ailments and appear to be safe even when placed in close proximity to a treated area for a prolonged period of time.

While actual soap fragments or pieces can be used as substance 20, an increased therapeutic effect has been observed when a scent oil extract of soap is used. A scent oil extract of the aforementioned Ivory soap was obtained. Gauze layer 12 was substantially saturated with the scent oil extract and used effectively as will be described in greater detail hereinafter. Thus, it is intended that the elements 20 shown in FIG. 1 can be fragments of soap within or on gauze layer 12, as well as droplets of scent oil held or absorbed by gauze layer 12.

Cover layer 14 is a substantially impermeable fabric or other material covering one side of gauze layer 12 and substance 20 contained therein. Cover 14 is slightly larger than gauze layer 12, and helps confine the scent, sublimates or vaporization products from substance 20 to the area upon which patch 10 is applied. Cover 14 can be provided with an adhesive coating for securing cover layer 14 to gauze layer 12.

Patch 10 can be used as thus far described, with the exposed side of gauze layer 12 placed against the skin, and cover layer 14 exposed on the outer side of patch 10. Patch 10 can be secured in the desired position on the skin using adhesive tape or other binding materials in a manner similar to the application and fixation of wound dressings.

For further integrity of patch 10, a transmission layer 22 can be used on the inwardly facing surface of gauze layer 12. In the exemplary embodiment, transmission layer 22 is a layer of breathable adhesive tape extending beyond gauze layer 12 to adhere to cover layer 14 about the periphery of gauze layer 12. Transmission layer 22 is a material through which the scent, sublimates or vaporization products of substance 20 pass readily. Thus, as illustrated in FIG. 2, gauze layer 12 containing substance 20 is confined within a pocket formed by substantially impermeable cover layer 14 on one face thereof and by permeable transmission layer 22 on the other side thereof.

In a further embodiment shown in FIG. 3 gauze layer 12, containing substance 20, is attached to an elongated web 30, which has tail end portions 32, 34 extending beyond opposite sides of gauze layer 12. The aforementioned transmission layer 22 of breathable tape can be used for attaching gauze layer 12 to web 30. Web 30 can be an adhesive tape or other substantially impermeable material functioning similarly to cover layer 14 for confining the scent from substance 20. Alternatively, a cover layer 14 can be interposed between gauze layer 12 and web 30.

Tails 32, 34 are used to facilitate securing gauze 12 in the desired position. Thus, tails 32, 34 can be provided with a coating of suitable adhesive on the surface thereof with gauze layer 12, so that tails 32, 34 can be adhered to the skin of a person using the present invention. The length, shape and other size of tails 32, 34 can be varied to provide patches 10 of different configurations for use on various parts of the human body. Further, elongated tails 32, 34 without adhesive also can be used. Web 30 can be in the nature of an elastic bandage or other binder material to wrap the leg, arm or torso of the person using it. Non-adhesive tails 32, 34 can be secured to each other or to the person by commonly used adhesive tape, mechanical fastener or other securing devices. As yet another alternative, tails 22 can be provided with complementary hook-and-loop components for securing one to the other.

In using patch 10, the area of pain, cramping or other discomfort is identified, and patch 10 is placed substantially centrally thereon to bring substance 20 in close proximity to the area to be treated. Patch 10 is secured in place via tape, tails 32, 34 or suitable means. Patch 10 should be kept on the area being treated for at least six to eight hours, or until such time as the discomfort subsides. While patch 10 can be worn substantially continuously, effective treatment of many discomforts has been observed when patch 10 is used for approximately six to eight hours during successive twenty-four hour periods. Thus, for many discomforts, effective treatments can be performed with nighttime applications of patch 10, while the user is resting or asleep and not overly active.

The present invention has been tried on a variety of individuals experiencing different types of pain, cramping and general discomfort. The observed effectiveness is reported below.

A marathon runner experienced frequent cramps, tenderness and generally achy sensations in calf muscles for more than ten months. Palpable knots in the calf muscle were present. Physical therapy treatments, including ultra-sound treatments, acupuncture and message provided little relief. Patch 10 was placed on the calf muscle. After two days, all symptoms were reported to be gone. During running a marathon, patch 10 was placed on the calf muscle of one leg, but not the other. The leg without patch 10 experienced pain, cramping and discomfort. The leg with patch 10 thereon experienced no similar symptoms.

An individual experienced increasing tightening and discomfort in the back shoulder area for five days. Patch 10 was applied for 2 successive evenings while sleeping. After two days all symptoms were gone.

An individual experienced severe myofascial pain (so called “trigger point pain”) and tightening in a shoulder for more than two years. Trigger point injections of local anesthetic, steroid treatment and botox injections provided no relief. Patch 10 was applied to the area. The person reported fifty-percent relief after one day and eighty-percent relief after two days, with all pain relieved after five days.

A second individual experienced myofascial pain in a shoulder for over one year following a sports-related injury. Standard treatments were not effective. After one week of inconsistent use of patch 10 fifty-percent relief of discomfort was reported.

A third individual experienced myofascial pain of a sharp burning sensation in a shoulder for thirteen months. Trigger point injections provided only short-term relief. Patch 10 was used sporadically, and ten-percent to twenty-percent relief was reported by the second day, and sixty-percent relief reported by the fifth day.

A fourth individual experienced myofascial pain between the shoulder blades for five days. Patch 10 was applied on the tender area for two successive evenings. All pain was reported to be gone.

An individual experienced severe neck and shoulder pain and tightness following radical neck surgery and radiation treatments for cancer. The pain and tightness were unresponsive to all treatments tried, including physical therapy, drugs, yoga, message, heating pads, hot soaks, weight training and the use of special apparatus. Patch 10 was applied to the area overnight. After five successive treatments pain and tightness relief of eighty-percent was reported. Continued relief was reported with continued applications of patch 10 overnight. After two weeks, all pain and discomfort was reported to be gone. After five months of continued, regular use of patch 10, pain and discomfort did not return.

An individual routinely experienced severe menstrual cramps. Patch 10 using scent oil was applied over the abdomen. Sixty-percent relief was reported.

An individual reported a long history of regular, severe menstrual cramps. Patch 10 attached to an elastic bandage was wrapped around the abdomen and worn at night. After two days forty-percent relief was reported.

An individual routinely experienced severe menstrual cramps. Patch 10 using scent oil was applied over the abdomen. Sixty-percent relief was reported.

An individual reported a long history of routine, severe menstrual cramps. Patch 10 attached to an elastic bandage was wrapped around the lower abdomen. After two days fifty-percent to sixty-percent relief was reported.

An individual reported severe menstrual cramps. Patch 10 was applied with a heating pad. After one day ninety-five percent relief was reported, and all pain was reported to be gone after the second day.

An individual reported experiencing severe menstrual cramps for many years. On a first day of severe, almost intolerable cramps, patch 10 was applied with initial significant relief. Patch 10 was worn continuously for two days, with one-hundred percent relief of cramping.

An individual suffering discomfort from a laparoscopic tubal ligation applied patch 10 over the umbilicus and reported generally feeling better.

An individual suffering from a herniated disc (L₃₋₄) reported persistent back pain for more than one year. Patch 10 using scent oil was applied, with reported pain relief of more than fifty-percent. Use was stopped after the second day due to a skin reaction, perhaps from the scent oil.

An individual with a herniated disc for three months had epidural steroid injections ten days apart, with only some relief. Patch 10 was applied and seventy-percent relief was reported initially. Pain and spasms were gone after two days.

An individual with a bulging disc for two months received acupuncture and chiropractic treatments with only temporary relief of pain. An epidural steroid injection provided no relief after three days. Patch 10 was applied with a heat pack for 30 minutes, and thereafter without heat. After three days fifty percent improvement was reported.

An 82 year old female with spinal stenosis reported back pain for several years. Epidural steroid injections provided some relief. Patch 10 was applied, and after three days seventy percent improvement was reported.

An 80 year old female with spinal stenosis reported many years of back pain. Patch 10 was applied, and complete relief of pain was reported.

An individual reported hip joint pain for several weeks. Patch 10 was applied to the buttock for two days, with initial reported pain relief of eighty-percent and subsequent total relief.

An individual reported irritable bowel syndrome for over fifty years, with worsening conditions the last five years. Symptoms included excessive gas, explosive bowel movements, frequent urge and bowel movements (seven or eight times daily), and very loose stools with frequent diarrhea. Daily treatments of prescribed and over-the-counter medicines, and dietary changes provided little relief. Patch 10 first with fragments 20 and later with scent oil 20 was applied over the lower abdomen while sleeping. Gradual relief of symptoms was reported over the first week. All other medications were stopped, and eighty-percent improvement in symptoms was reported. Gas, looseness of stools and diarrhea were gone, and bowel movement urges were reduced to three or four daily.

An individual with digestive problems for more than forty years had stomach cancer surgery four years ago. Since then recurrent diarrhea and gas problems were experienced. Patch 10 was applied over the abdomen, with improvement in conditions realized after two days. After ten days of use, seventy-percent improvement was reported.

A fifty-six year old female exhibited a slight limp and reported pain in the area of scar tissue from a severe infection of the calf muscle at age ten. For more than twenty years, pain medication was taken orally or methyl salicylate patches were applied to the area. Patch 10 was applied, and after three days the pain was gone.

A fifty year old male reported stiffness in the neck upon awakening. Patch 10 was applied over the affected area. Within one hour the condition lessened and was completely relieved within one day. The relief was only temporary, and stiffness returned on the second day. The patch was repositioned to an area seemingly more affected, and further relief was experienced.

Patch 10 has manifested effectiveness in treating occurrences of muscle cramping, muscle spasms, nerve pain, pinched nerves and discomfort from a number of sources and conditions. It is believed that other pains, discomforts, spasms and cramps might be equally responsive to treatment by patch 10. Conditions that respond to the present invention appear to be those for which relief can be obtained by treating the symptom without treatment of the underlying condition causing the symptom, with the condition being treated otherwise, or simply healing or entering states of remission. It is anticipated that other conditions for which the present invention will provide effective treatment would include various intractable, otherwise difficult to treat painful conditions including sympathetic dystrophy, interstitial cystitis and fibromyalgia. On the other hand the present invention has not been demonstrative as effective in treating discomfort and pain associated with degenerative rheumatoid arthritis, severe inflammation, tendinitis, or torn muscles.

The mechanism of treatment is not completely understood; however, there appears to be a relationship to the scent or components thereof and an apparent transdermal transmission thereof to the area being treated. Several users have reported an improved therapeutic result when a concentrated scent extract of soap was used as substance 20, when compared to similar treatments using fragments of soap, leading to a belief that components of scent are involved. However, other components may be responsible for the therapeutic effect, including, potentially, products resulting from the sublimation of substance 20. These can include the products resulting from the saponification of fat and alkali in making soap, or remaining non-reacted fat or alkali. Further, something other than a transdermal transfer may occur. Whatever the mechanism, a significant therapeutic result has been achieved by placing substance 20 in close proximity to the area to be treated and maintaining the close proximity position for an extended time, such as about at least six to eight hours during a twenty-four hour time period.

Analyzing the levels of relief reported by individuals who had various different conditions has lead to an observation of three categories of conditions and the effectiveness of treatments with the present invention. Conditions of pain or discomfort associated with dysfunctions not related to damage of nerves or other tissue are highly responsive to treatment with the present invention, and have responded with rapid, almost total relief. Conditions of pain or discomfort that include moderate tissue damage or abnormal surrounding tissue, such as moderate inflammation respond to treatment to a lesser degree. The discomfort can be controlled, allowing time for the underlying condition to heal. Also in this category are conditions of less severe mechanical or anatomic changes, for which some relief of discomfort can be achieved. Only conditions that include obvious, severe mechanical or anatomic changes, such as end stage arthritis, severe inflammations, torn ligaments and the like have not been helped significantly by the present invention.

The aforedescribed uses by individuals supports at least several conclusions. Soap has a therapeutic effect for several conditions when the soap is placed in close proximity to the affected area and retained there for an extended time period. Components of soap scent have a therapeutic effect when applied transdermally to the affected area. Medications can be administered transdermally to treat internal organs. These conclusions, and particularly the indications that internal organs can be treated by the transdermal administration of medications, has implications for application with other drugs and medications beyond the use of soap and soap products as described herein.

Variations and modifications of the foregoing are within the scope of the present invention. It is understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention. The claims are to be construed to include alternative embodiments to the extent permitted by the prior art.

What is claimed is:

1. A method for treating human physical discomforts, comprising:
locating the area of discomfort;
obtaining a substance that emits the scent of the product from the saponification of fat and alkali;
placing the substance in close proximity to the area of discomfort; and
maintaining the substance on the area of discomfort.

2. The method of claim 1, including sandwiching the substance between the area of discomfort and a cover of material that is relatively impermeable.

3. The method of claim 2, including interposing a layer of permeable material between the area of discomfort and the substance.

4. The method of claim 1, including interposing a layer of permeable material between the area of discomfort and the substance.

5. The method of claim 1, said step of obtaining a substance including obtaining fragments of soap made from fat and sodium hydroxide and of a type for cleansing human skin.

6. The method of claim 5, including interposing a layer of permeable material between the area of discomfort and the fragments of soap.

7. The method of claim 1, said step of obtaining a substance including obtaining a scent oil extract of soap made from fat and sodium hydroxide and suitable for cleansing human skin; and said method further comprising providing an absorbent layer and applying the scent oil to the absorbent layer.

8. The method of claim 7, including interposing a layer of permeable material between the area of discomfort and the layer of absorbent material.

9. The method of claim 7, including sandwiching the absorbent layer between a layer of permeable material and a cover of material that is relatively impermeable.

10. A transdermal patch for the treatment of human physical discomforts, comprising:
an inner layer adapted for placement in close proximity to human skin;
a substance in said inner layer emitting the scent of soap made from fat and sodium hydroxide and suitable for cleansing human skin; and
an outer layer of material on said inner layer for protecting said inner layer and said substance.

11. The patch of claim 10, said outer layer being substantially impermeable to the transmission therethrough of said scent of said substance.

12. The patch of claim 11, including providing a transmission layer on a face of said inner layer opposite said outer layer.

13. The patch of claim 11, including providing a web having tails extending in opposite directions beyond said inner layer.

14. The patch of claim 10, said substance being fragments of soap.

15. The patch of claim 10, said substance being a scent oil extract of soap.

16. A method for treating human physical discomfort comprising steps of:
identifying on a person the area in which discomfort is experienced;
administering the scent of common soap transdermally to the area of discomfort; and
continuing the transdermal administration of the scent of soap for regular prolonged intervals until the discomfort subsides.

17. The method of claim 16, including repeating the transdermal administration of soap scent upon the recurrence and/or re-intensification of discomfort.

18. The method of claim 16, including continuing the transdermal administration of soap scent for a continuous period of at least about six hours.

19. The method of claim 18, including repeating the transdermal administration of soap scent for a continuous period of at least about six hours during successive twenty-four hour time periods.

20. A method for treating muscle cramps and muscle spasms comprising steps of:
identifying on a person the area in which at least one of muscle cramps and muscle spasms are experienced;
administering the scent of common soap transdermally to the area identified; and
continuing the transdermal administration of the scent of soap for regular prolonged intervals.

21. A method for treating pain comprising steps of:
identifying on a person the area in which pain is experienced;
administering the scent of common soap transdermally to the area of pain; and
continuing the transdermal administration of the scent of soap for regular prolonged intervals.

22. A method for treating menstrual cramps comprising steps of:
administering the scent of common soap transdermally to the lower abdomen; and
continuing the transdermal administration of the scent of soap for regular prolonged intervals.

23. A method for relieving the symptoms of irritable bowel syndrome, comprising steps of:
administering the scent of common soap transdermally to the lower abdomen; and
continuing the transdermal administration of the scent of soap for regular prolonged intervals.

24. A method for treating myofascial pain comprising steps of:
identifying on a person the area in which the pain is experienced;
administering the scent of common soap transdermally to the area of pain; and
continuing the transdermal administration of the scent of soap for regular prolonged intervals.

25. A method for treating pain from a pinched nerve comprising steps of:
identifying on a person the area in which the pinched nerve has occurred;
administering the scent of common soap transdermally to the area identified; and
continuing the transdermal administration of the scent of soap for regular prolonged intervals.

26. A method for treating discomfort in a person, including pain, spasms and cramps, comprising:
identifying the area of discomfort;
obtaining at least one of soap made from fat and sodium hydroxide and a substance having the scent of soap made from fat and sodium hydroxide;
placing the at least one of soap made from fat and sodium hydroxide and a substance having the scent of soap made from fat an sodium hydroxide in close proximity to the area of discomfort; and
maintaining the at least one of soap made from sodium hydroxide and a substance having the scent of soap made from sodium hydroxide in close proximity to the area of discomfort for at least about six hours in a twenty-four hour period.

27. A method for treating diarrhea and intestinal gas, including those caused by irritable bowel syndrome, comprising:
obtaining a substance including at least one of soap made from fat and sodium hydroxide, a substance having the scent of soap made from fat and sodium hydroxide and products occurring from the saponification of fat and alkali;
placing the substance in close proximity to the lower abdomen; and
maintaining the substance in close proximity to the lower abdomen for at least about six hours in a twenty-four hour period.

28. A method for treating pain from spinal stenosis, comprising:
identifying the area of pain along the spine;
obtaining a substance including at least one of soap made from fat and sodium hydroxide, a substance having the scent of soap made from fat and sodium hydroxide and products occurring from the saponification of fat and alkali;
placing the substance in close proximity to the area of pain; and
maintaining the substance in close proximity to the area of pain for at least about six hours in a twenty-four hour period.

29. A method for treating muscle stiffness, including stiffness of neck muscles, comprising:
identifying the area of muscle stiffness;
obtaining a substance including at least one of soap made from fat and sodium hydroxide, a substance having the scent of soap made from fat and sodium hydroxide and products occurring from the saponification of fat and alkali;
placing the substance in close proximity to the area of muscle stiffness; and
maintaining the substance in close proximity to the area of muscle stiffness for at least about six hours in a twenty-four hour period.

30. A method for treating pain from scar tissue, including scar tissue from healed injuries, wounds, surgical wounds and radiation therapy, comprising:
identifying the area of pain in a scar;
obtaining a substance including at least one of soap made from fat and sodium hydroxide, a substance having the scent of soap made from fat and sodium hydroxide and products occurring from the saponification of fat and alkali;
placing the substance in close proximity to the painful area of the scar; and
maintaining the substance in close proximity to the painful area of the scar for at least about six hours in a twenty-four hour period.

31. A method for treating pain from a bulging spinal disc, comprising:
identifying the area of the bulging disc;
obtaining a substance including at least one of soap made from fat and sodium hydroxide, a substance having the scent of soap made from fat and sodium hydroxide and products occurring from the saponification of fat and alkali;
placing the substance in close proximity to the bulging disc; and
maintaining the substance in close proximity to the bulging disc for at least about six hours in a twenty-four hour period.

32. A method for treating internal organs, comprising:
identifying the organ to be treated;
placing soap in close proximity to an area of skin covering the organ; and
retaining the soap in the area for at least about six hours during a twenty-four hour period.

33. A method for treating internal organs, comprising:
identifying the organ to be treated;
selecting a medication with which to treat the organ;
placing the medication in close proximity to an area of skin covering the organ to be treated; and
retaining the medication in close proximity to the area of skin for an extended continuous period of time.
